(19) United States
(12) Patent Application Publication
(10) Pub. No.: US 2008/0109026 A1
(43) Pub. Date: May 8, 2008

(54) Apparatus and Methods for Performing Brain Surgery

(75) Inventor: Amin Kassam, Pittsburgh, PA (US)

Correspondence Address: David M Carter;CARTER DELUCA FARRELL & SCHMIDT, Suite 225, 445 Board Hollow Road, Melville, NY, 11747, US

(73) Assignee: STRATEGIC TECHNOLOGY ASSESSMENT GROUP, Pittsburg, PA (US)

(21) Appl. No.: 11/665,666

(22) PCT Filed: Oct. 28, 2005
(86) PCT No.: PCT/US05/39185

§ 371 Date: Apr. 18, 2007

Related U.S. Application Data
(60) Provisional application No. 60/623,094, filed on Oct. 28, 2004.

(51) Int. Cl. A61B 17/00; A61B 1/00; A61M 29/00; A61M 5/178

(52) U.S. Cl. 606/190; 600/101; 604/164.11; 606/191

(57) ABSTRACT

Less invasive surgical techniques for performing brain surgery are disclosed in which a dilating obturator and cannula assembly is inserted into brain tissue until the obturator tip and cannula are adjacent the target tissue. The obturator is removed and surgery is performed through the cannula. In preferred embodiments the obturator and cannula are placed using image guidance techniques and systems to coordinate placement with pre-operative surgical planning. A stylet with associated image guidance may he inserted prior to insertion of the obturator and cannula assembly to guide insertion of the obturator and cannula assembly. Surgery preferable is performed using an endoscope partially inserted into the cannula with an image of the target tissue projected onto a monitor.

### PRIORITY

This application claims the benefit of the priority of co-pending U.S. Provisional application 60/623,094, filed Oct. 28, 2004.

### TECHNICAL FIELD

The present disclosure relates to methods of accessing and performing surgery within the brain.

### BACKGROUND

Diagnosis and treatment of conditions affecting the brain are among the most difficult and complex problems that face the medical profession. The brain is a delicate soft tissue structure that controls bodily functions through a complex neural network connected to the rest of the body through the spinal cord. The brain and spinal cord are contained within and protected by significant bony structures, e.g., the skull and the spine. Given the difficulty of accessing the brain through the hard bony protective skull the diagnosis and treatment of brain disorders presents unique challenges not encountered elsewhere in the body.

Diagnosis of brain disorders requires clear, accurate imaging of brain tissue through the skull. In recent years significant advances have been made in imaging technology, including stereotactic X-ray imaging, Computerized Axial Tomography (CAT), Position Emission Tomography (PET) and Magnetic Resonance Imaging (MRI). See, for example, Butler U.S. Pat. No. 6,359,959. These imaging devices and techniques permit the surgeon to examine conditions within the brain in a non-invasive manner without opening the skull. If a target lesion or mass is identified through use of one or more imaging techniques, it may be necessary or desirable to biopsy a lesion within the brain. Stereotactic techniques and apparatus for directing a biopsy needle to the site are described and shown, for example, in Cosman U.S. Pat. Nos. 6,331,180 and 6,416,520.

Once a diagnosis has been reached based upon one or more imaging techniques, a treatment plan must be developed. One available method of treatment involves X-ray therapy such as disclosed in Leber U.S. Pat. No. 5,513,238; Shiu U.S. Pat. No. 5,555,283; Cosman U.S. Pat. Nos. 5,748,703, 5,778,043, 5,947,981, 6,459,769; and Kooy U.S. Pat. Nos. 6,005,919, 6,041,101, and 6,278,766. Alternatively, surgical treatment may be necessary or desired. In order to operate surgically on the brain, access must be obtained through the skull and delicate brain tissue containing blood vessels and nerves that can be adversely affected by slight disturbances. Therefore, great care must be taken in operating on the brain not to disturb delicate blood vessels and nerves so that adverse consequences do not result during or after surgery. Brain surgery can be highly invasive. In some instances, in order to obtain access to target tissue, a substantial portion of the skull is removed and entire sections of the brain are retracted to obtain access. Of course, such techniques are not appropriate for all situations, and not all patients are able to tolerate and recover from such invasive techniques. It is also known to access certain portions of the brain by forming a hole in the skull, but only limited surgical techniques may be performed through such smaller openings. In addition, some techniques have been developed to enter through the nasal passages, opening an access hole through the occipital bone to remove tumors located, for example, in the area of the pituitary.

A significant advance in brain surgery is stereotactic surgery involving a stereotactic frame correlated to stereotactic X-ray images to guide a probe or other surgical instrument through an opening formed in the skull through brain tissue to a target lesion or other body. See, for example, U.S. Pat. Nos. 6,331,180 and 6,416,520. A related advance is frameless image guidance, in which an image of the surgical instrument is superimposed on a pre-operative image to demonstrate the location of the instrument to the surgeon and trajectory of further movement of the probe or instrument. Image guided surgery is described, for example, in Guthrie U.S. Pat. Nos. 5,230,623, 5,971,997, 6,120,465, and 6,409,686; Cosman U.S. Pat. Nos. 5,662,111, 5,848,967, 6,006,126, 6,167,295, 6,259,943, 6,275,725, 6,351,661, 6,405,072, 6,662,036, and 6,675,040; and Faro U.S. Pat. Nos. 5,251,127, 5,305,203, and 5,748,767.

Notwithstanding the foregoing advances in imaging technology and both frame and frameless stereotactic image guidance techniques, there remains a need for improved surgical techniques and apparatus for operating on brain tissue.

### SUMMARY OF THE DISCLOSURE

Apparatus and methods are disclosed for atraumatically dilating brain tissue to access target tissue within the brain.

A preferred apparatus for accessing brain tissue has a dilating obturator with a blunt distal tip, a substantially cylindrical shaft portion, and a proximal handle portion. A cannula is disposed around the shaft portion and preferably is made of a transparent material. The obturator and cannula assembly preferably is associated with an image-guided surgery system so that placement of the obturator and cannula assembly can be carefully monitored and controlled as the obturator and cannula assembly is atraumatically inserted into brain tissue.

In a first embodiment the obturator has a longitudinal channel therethrough configured and dimensioned to receive the shaft of a narrow stylet or probe. The stylet or probe has attached thereto image guidance means calibrated to indicate the orientation and position of the stylet or probe. An image guidance system interacts with the stylet or probe to display for the surgeon on a monitor an image of the stylet or probe superimposed onto an image of the patient s brain, such as an MRI image. The image may be a pre-operative MRI image used for surgical planning. When the stylet or probe is mounted in the longitudinal channel of the obturator, the superimposed image of the probe also is indicative of the position and orientation of the dilating obturator and the cannula.

Traditional methods are used to incise and retract soft tissue of the scalp covering the skull. A hole is made in the skull, and the dura is opened and retracted to provide access to the brain. In the first method, the stylet or probe is inserted through the obturator longitudinal channel and advanced until a length of the stylet or probe extends out of and beyond the blunt tip of the dilating obturator. The dilating obturator and cannula assembly is held back away from the tissue as the stylet or probe is gently advanced through the brain tissue under both direct vision and positional image guidance until the tip of the stylet or probe is adjacent the target tissue. Once the stylet or probe is placed and the position is confirmed using the image guidance system, the blunt dilating obturator and cannula assembly is slowly and carefully advanced into the brain tissue to atraumatically spread the tissue over the dilating tip and around the cannula while maintaining the position of the stylet or probe as a guide to advancement of the obturator and cannula assembly. A gentle back and forth rotation during insertion may facilitate placement of the obturator cannula assembly. Once the dilating obturator and cannula assembly are correctly positioned adjacent the target tissue, the stylet or probe and dilating obturator are removed, leaving the cannula in place to support and protect the dilated brain tissue. Preferably, the cannula is clear so that the dilated brain tissue may be visually inspected through the walls of the cannula to assure that no damage was caused to surrounding brain tissue during insertion of the device.

Alternatively, in a second contemplated method the image guidance means may be mounted directly to the dilating obturator and cannula assembly so that the obturator and cannula assembly may be inserted without a separate stylet or probe. In this configuration, the obturator and cannula assembly is inserted into the brain tissue under image guidance until the obturator is adjacent the target tissue. Once the obturator and cannula assembly is positioned, the dilating obturator is removed, leaving the cannula in place.

In yet a third contemplated method, the dilating obturator and cannula assembly may be inserted into the brain under direct visualization without use of an image guidance system.

After the cannula is placed, surgery may be performed through the cannula, either under direct vision or more preferably using an endoscope and camera system to project an enlarged image of the target tissue onto a monitor to visualize the tissue during surgery.

Preferably, the cannula has a diameter of approximately 10 mm to 20 mm, and more preferably 10 mm to 15 mm. An endoscope of a substantially smaller diameter, such as a 4 mm endoscope, is mounted partially inserted into the cannula. The endoscope is mounted to one side of the cannula and inserted so that the image projected onto the monitor is of the target tissue at the end of the cannula. In practice, a 4 mm endoscope inserted approximately halfway into the cannula is appropriate to create the desired image display while leaving a substantial portion of the cannula open and available for the insertion of instruments to perform surgery. Optionally, a camera holder may be used to secure the endoscope in the desired position.

Appropriate surgical instruments are then used to perform surgery upon the target tissue. For example, scissors, graspers and suction tools may be inserted through the cannula, visualizing the tips of the instruments to perform the desired procedure either directly with the naked eye or through a microscope, or indirectly through the endoscope using the endoscope eyepiece or more preferably and camera system to display the image on a monitor. A preferred instrument for debulking brain tissue is a fluidized ultrasonic instrument, such as CUSA (Valleylab, Boulder Colo.) During surgery, monitoring equipment may be used to monitor brain function during surgery to assist the surgeon in understanding the effects of the actions taken during surgery operating on the brain so that the surgery may be terminated in the event an indication of an adverse effect is detected.

After surgery upon the target tissue is complete, the cannula is gently removed, and the dura, skull and scalp are closed in a traditional fashion.

The devices and methods disclosed herein provide numerous advantages in performing brain surgery. Gentle atraumatic dilation of the brain tissue makes it possible to operate further inside the brain than otherwise would be possible utilizing traditional surgical techniques. The disclosed methods and apparatus create an access area to work while simultaneously protecting adjacent brain tissue from inadvertent collateral damage and trauma that might otherwise occur if more traditional surgical techniques were to be utilized. In addition, accessing target tissue through the cannula as contemplated avoids more invasive techniques that involve removing substantial portions of the skull and retracting large portions of the brain to gain access to operate on target tissues. In some cases, the devices and methods may make it possible to operate on target tissue that would, without these devices and methods, otherwise be regarded as inoperable using previously known techniques.

### BRIEF DESCRIPTION OF THE DRAWINGS

The accompanying drawings form a part of this disclosure, in which:

FIG. 1 is a perspective view, with parts separated, of an access device in accordance with a first embodiment;

FIG. 2 is a cross-sectional view of an obturator and cannula assembly, with parts separated, in accordance with the first embodiment;

FIG. 3 is a perspective view of an obturator, cannula and stylet assembly in accordance with the first embodiment;

FIG. 4 is a perspective view, with parts separated, of an access device in accordance with a second embodiment;

FIG. 5 is a perspective view, with parts separated, of an access device in accordance with a third embodiment;

FIG. 6 is an illustration of the first embodiment with the stylet inserted to a point adjacent target tissue within the brain;

FIG. 7 is an illustration of the first embodiment with the obturator and cannula assembly partially inserted into and atraumatically separating brain tissue;

FIG. 8 is an illustration of the first embodiment, with the obturator and cannula assembly inserted over the stylet to target tissue;

FIG. 9 is an illustration of a cannula in place holding brain tissue apart to provide access to target tissue;

FIG. 10 is an illustration of an endoscope mounted partially within the cannula and an ultrasonic dissection tip inserted to debulk and remove target tissue;

FIG. 11 is an illustration of a cannula in place after target tissue has been removed; and

FIG. 12 is an illustration showing brain tissue having resumed its position occupying the space previously occupied by the cannula during surgery.

### DETAILED DESCRIPTION OF THE PREFERRED EMBODIMENTS

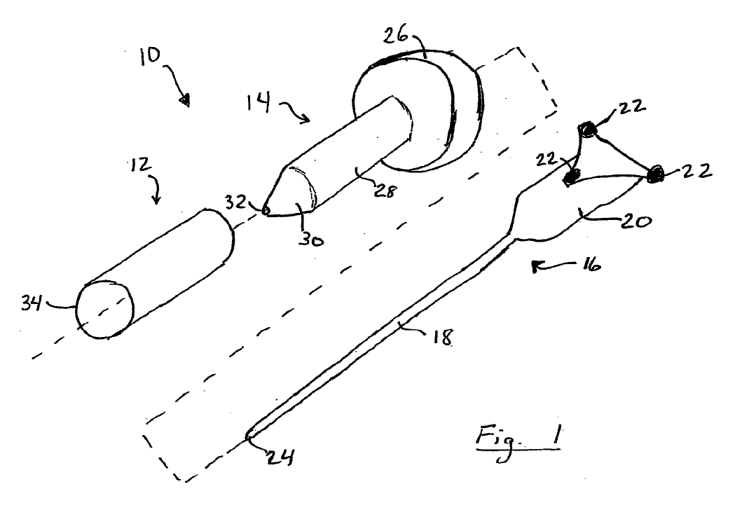

Referring now to the drawings, FIG. 1 illustrates a first embodiment of an apparatus 10 for accessing target tissue within the brain in order to perform brain surgery. The access device includes a cannula 12, a dilating obturator 14 and a stylet or probe 16. Stylet or probe 16 has a small diameter elongated shaft 18, a handle 20 and associated position indicators 22 for an image guidance system. Stylet shaft 18 has a blunt tip 24 that can be inserted into and advanced through brain tissue. In FIG. 1, image guidance position indicators are shown as infrared reflectors of the type use in connection with optical image guidance systems. As shown, the infrared reflectors used with such a system are mounted to the stylet handle in a customary triangular configuration calibrated to identify the tool to the image guidance system. Such imaging systems are available, for example Medtronic Surgical Navigation Technologies (Denver, Colo.), Stryker (Kalamazoo, Mich.), and Radionics (Burlington Mass.).

Typically, the positioning of the indicator balls is calibrated such that the image guidance system can project an image of the tool onto a display of images of the patient s brain, such as MRI images used to plan surgery. Calibration of instruments to an image guidance system is disclosed, for example, in Costales U.S. Pat. No. 5,921,992. As the instrument is inserted, the surgeon can see the relative position of the instrument relative to the structures of the brain as reflected on images used to plan surgery, and particularly with respect to the target tissue.

Dilating obdurator 14 has a proximal handle portion 26, a substantially cylindrical shaft portion 28, and a blunt conical dilating tip 30. A longitudinal access channel 32 extends through the dilating obturator 14. The longitudinal channel is configured and dimensioned to receive therethrough the shaft 18 of the stylet or probe 16. Cannula 12 is substantially cylindrical and is configured to slide over and mount onto the substantially cylindrical shaft 28 of the dilating obturator 14. Leading edge 34 of cannula 12 may be chamfered to reduce insertion force and minimize trauma during insertion into the brain.

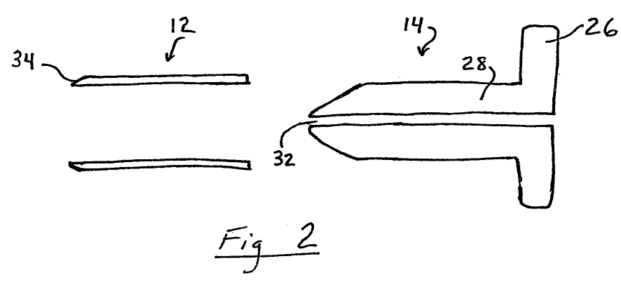

FIG. 2 is a cross-section view of the cannula 12 and dilating obturator 14 of the first embodiment, illustrating the chamfered lead edge 34 of cannula 12 and the longitudinal access channel 32 extending axially through the entire length of the dilating obturator.

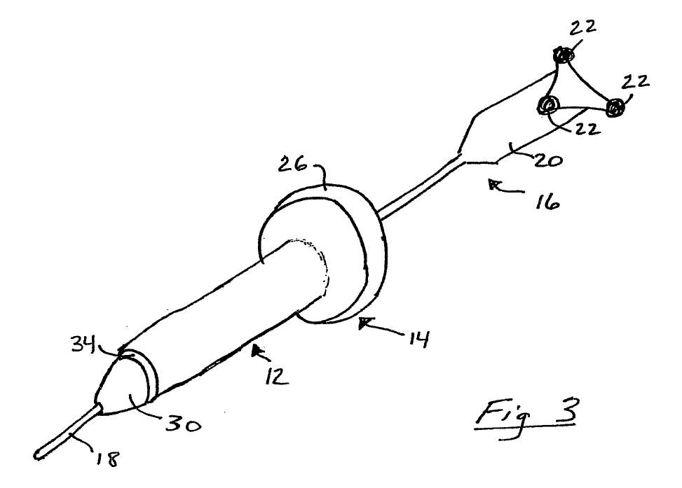

FIG. 3 is a perspective view of the first embodiment in an assembled condition, with cannula 12 disposed over the shaft of dilating obturator 14 with stylet or probe shaft 18 inserted through the longitudinal access channel of the dilating obturator and projecting from the distal, conical tip of the obturator.

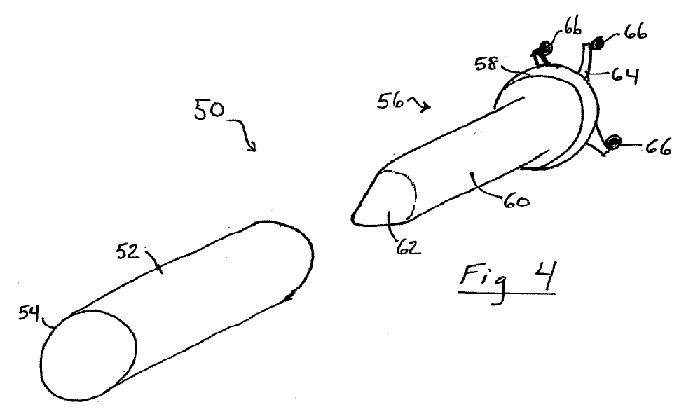

FIG. 4 is a perspective view, with parts separated, of a second embodiment of an access device 50 for brain surgery. Access device 50 includes a cannula 52 with a chamfered lead edge 54. Access device 50 also includes a dilating obturator 56 having a handle portion 58, a substantially cylindrical shaft 60 and an atraumatic blunt dilating tip 62. Preferably, blunt tip 62 is conical with a rounded distal end. Cannula 12 is configured and dimensioned to mount over shaft 60 of the dilating obturator. The obturator shaft is configured and dimensioned to removably fit into the cannula inner diameter and to occupy the open space within the cannula. As shown in FIG. 4 in the second embodiment, the image guidance identification device 64 with infrared imaging reflectors 66 is attached directly to the dilating obturator, eliminating the stylet or probe of the first embodiment. Accordingly, the dilating obturator of the second embodiment also need not include the longitudinal access channel for the stylet or probe.

The devices of the invention disclosed herein are shown for illustration purposes with infrared reflectors as used with available optical image guidance systems. Other guidance systems, such as magnetic or electromagnetic or radio transmitting systems may also be used, and the illustration of infrared reflectors and discussion of optical image guidance systems are exemplary only and are not intended to be limiting. In addition, currently available image guidance systems superimpose an image of the tool onto a pre-operative image. It is contemplated that as technology continues to progress that real-time imaging capability may become available in the operating room, and that the image of the tool may then b e shown in relation to the surrounding tissue structures on a real time image.

FIG. 5 is a perspective view, with parts separated, of a third embodiment of an access device 70 for brain surgery. Access device 70 includes cannula 72 with chamfered lead edge 74, and a dilating obturator 76. Dilating obturator 76 includes a handle 78, substantially cylindrical shaft 80 and dilating tip 82, which is preferably conical with a rounded distal tip. Access device 70 does not include apparatus for calibrating the position of the dilating obturator with an image guidance system or a stylet or probe for aiding insertion of the dilating obturator.

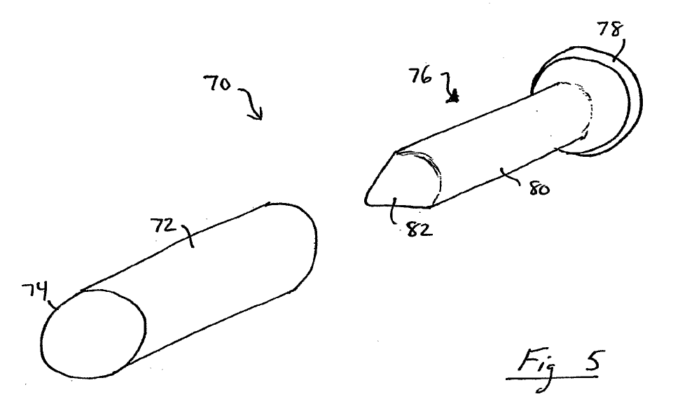

FIGS. 6-10 illustrate the use of the access device 10 of the first embodiment during minimally invasive brain surgery, as will now be described.

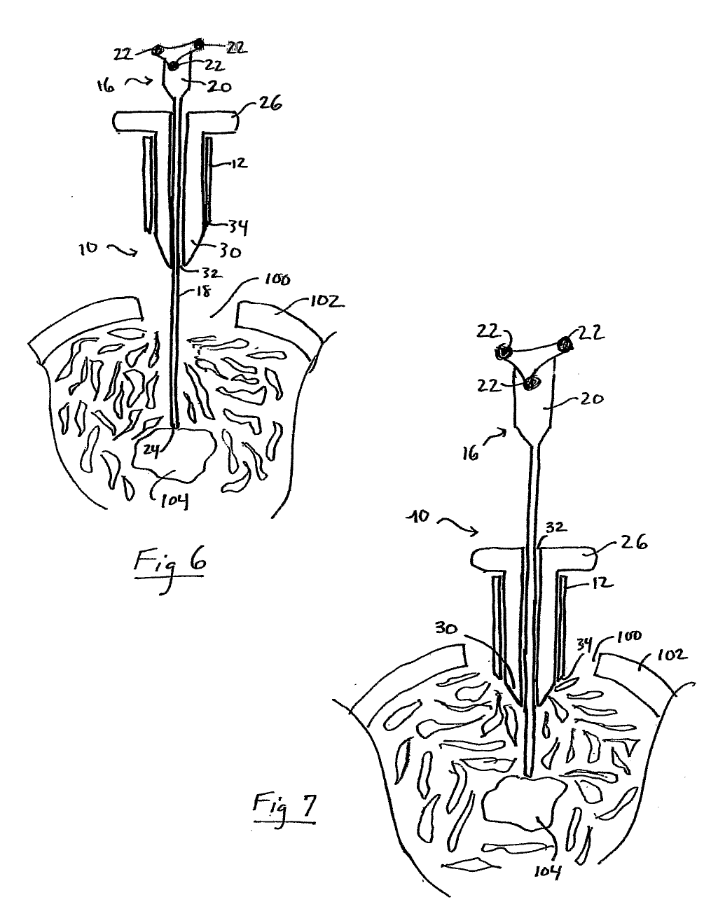

In FIG. 6, a partial cross-section view of the access device 10 with stylet shaft 18 of stylet 16 inserted through an opening 100 formed in a patient s skull 102 through brain tissue until tip 24 of stylet 28 is adjacent target tissue 104. Opening 100 is made in a traditional manner, by incising the prepared and marked scalp, dissecting the scalp away from the underlying bony skull 102, retracting the scalp away from the area where hole 100 is to be formed, and then forming hole 100 using a drill, saw or similar apparatus in a known manner. After an opening has been formed in the skull, the dura overlying and protecting the brain is carefully incised and retracted to provide access to the brain. Stylet 16 is approximately 12 cm to 15 cm in length and approximately 3 mm in diameter and may be atraumatically urged through brain tissue until the target tissue is reached. Because stylet handle 20 is associated with imaging targets 22 the position of the stylet may be confirmed one or more times during insertion again st pre-operative surgical planning images using an image guidance system. As shown in FIG. 7, once stylet 16 is placed, the dilating obturator with cannula is advanced carefully along the stylet so that the blunt conical tip 30 atraumatically dilates the brain tissue. In FIG. 7, the dilating obturator is shown partially inserted into the brain, with blunt conical tip 30 spreading the brain tissue as the obturator is advanced.

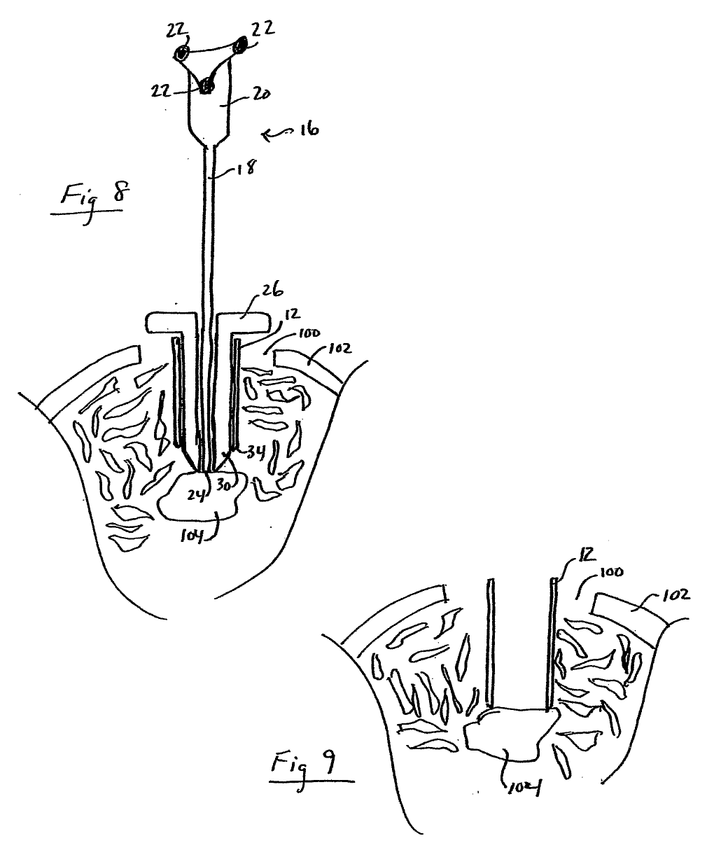

In FIG. 8, the access device is shown inserted into brain tissue until the tip of the dilating obturator 30 is adjacent the target tissue. As shown, the brain tissue has been spread apart and surrounds cannula 12. With the access device fully inserted, the stylet and obturator are removed, leaving the open cannula 12 to provide surgical access to the target tissue, as illustrated in FIG. 9. The brain tends to occupy the space available within the skull and, as shown in FIG. 9, after the obturator is removed the target tissue will have a tendency to approach the open end of the cannula. If this does not naturally occur it may be desirable to separately advance the cannula forward either before or after removing the dilator so that the end of the cannula is located directly next to the target tissue.

After the access cannula is placed as shown in FIG. 9, surgery may be performed upon the target tissue through the cannula. In this regard, it is contemplated that cannula lengths of up to about 6 cm may be necessary or desirable, although a cannula length of about 4 cm should be sufficient to reach most areas of the brain where surgery is to be performed using the access device and methods described herein. It is also contemplated that the cannula may have an inner diameter of approximately 10 mm to 20 mm, and more preferably about 10 mm to 15 mm to allow multiple instruments, such as graspers, dissectors, scissors, and suction instruments to be inserted through the cannula to perform surgery. The cannula wall thickness may be on the order of from about 1 mm to about 3 mm. In the event removal of tissue is desired, a debulking suction irrigation device such as a CUSA device (Valleylab, Inc., Boulder Colo.) may be used. See, for example, Rose U.S. Pat. No. 6,083,191; Stoddard U.S. Pat. Nos. 6,214,017, 6,256,859, and 6,654,999; and Cimino U.S. Pat. No. 6,602,227. Alternatively, a scissor and separate suction tube may be used.

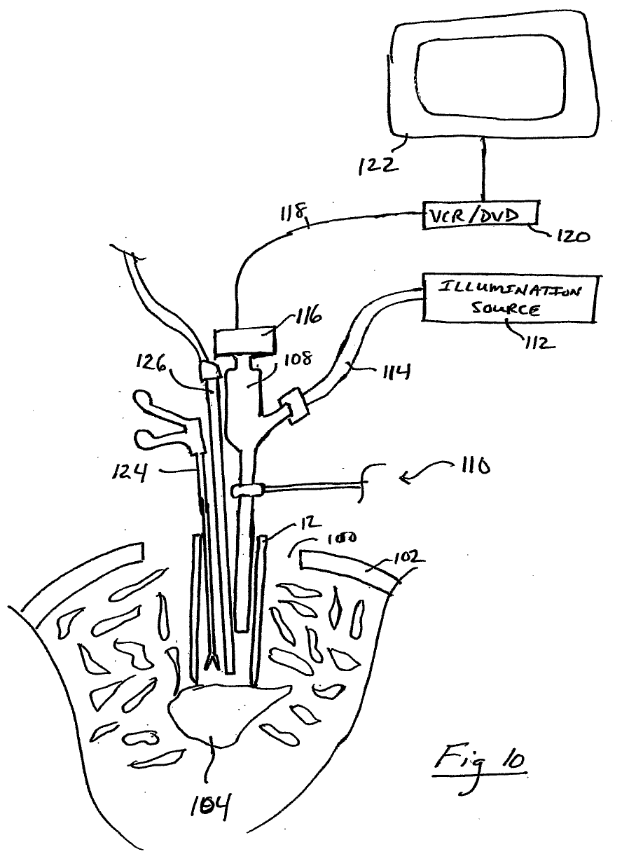

In a preferred method of performing surgery illustrated in FIG. 10, an endoscope of approximately 4 mm diameter is partially inserted and held to one side of the cannula, and the image of the end of the cannula and the target tissue is projected onto a monitor for viewing by the operating surgeon, assistants and others. Advantageously, a recording of the surgery also may be made. In FIG. 10, the endoscope 108 is illustrated inserted into the cannula 12 and held in place by the arm 110 of a scope holding device, thereby eliminating the need for the surgeon or assistant to hold the scope. Endoscope 108 is attached to a source of illumination 112 by a light cable 114. While the endoscope can be used under direct vision utilizing the endoscope eyepiece, it is preferred to attach a camera 116 to the endoscope which in turn is attached via a cable 118 to a video device 120 such as a VCR or DVD with an accompanying monitor display 122. Recent advances in operating room display equipment permit large monitoring devices, such as flat panel displays to be used. The latter display is particularly useful for teaching or lecturing purposes, as it allows multiple persons to observe the surgical technique. Without such a display, it would be impractical to have numerous persons in the operating field attempting to observe the surgery. Live telesurgery also is contemplated.

Also shown in FIG. 10 is a scissor 124 and suction tube 126 being used to debulk and remove target tissue 104. Preferably, at all times during insertion of the access device into the brain and during surgery through the cannula, the patient's brain function and condition is monitored so that the surgeon may be alerted in the event the patient becomes distressed or otherwise is adversely affected by the surgeon's actions. In the event signs of stress or adverse effects are noted, the surgeon may decide to continue the surgery, wait to see if the patient stabilizes, or terminate the procedure. Because of the sensitive nature of brain tissue and associated nerves and blood vessels, it is not uncommon for a surgeon to terminate a procedure before removing all target tissue in order to avoid the risk of serious adverse effects upon the patient.

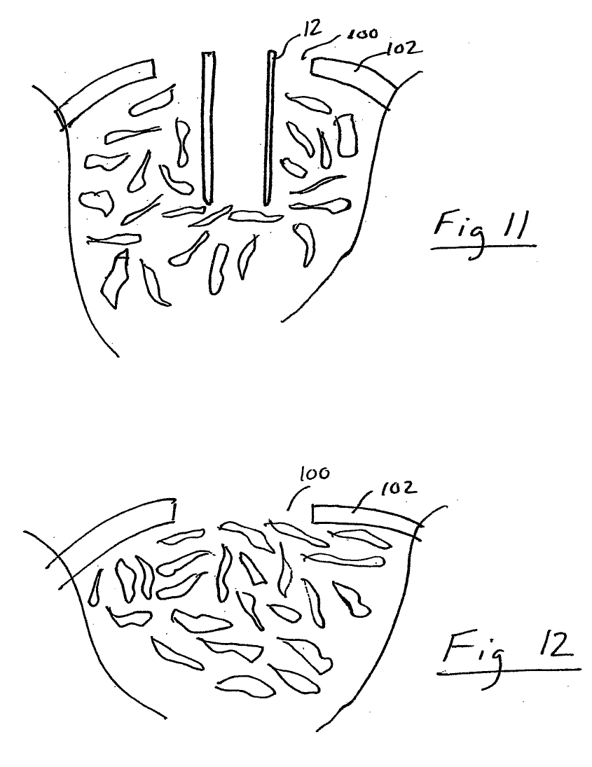

After surgery on the target tissue is complete, the instruments are removed from the cannula. As the target tissue is removed, the brain tissue fills the void formed by removing the target tissue so that healthy brain tissue underlying the now removed target tissue is adjacent the end of the cannula 12, as shown in FIG. 11. The cannula is then gently removed and the brain tissue naturally fills the space formerly occupied by the cannula, as shown in FIG. 12. This can take several minutes, but is relatively atraumatic. The dura, skull and scalp are closed in a known manner.

It is contemplated that the cannula may be from about 2 cm to about 6 cm in length, although different lengths may be desirable for particular situations. The cannula also preferably is clear, and is made to have a smooth outer surface to minimize trauma to the brain tissue. An important function of the cannula is to maintain the brain tissue in E separated condition to provide access and room to perform surgery. Just as important, however, is the function of the cannula to protect surrounding brain tissue from trauma due to contact with instruments during surgery. Thus, the cannula performs the dual functions of maintaining working space created during insertion of the obturator and cannula assembly and protecting surrounding brain tissue from trauma that might otherwise be caused during surgery by contact with surgical instruments. Alternate cross-sectional shapes for the cannula and obturator also are contemplated, such as square, oval, or elliptical. Of course, the dilating tip configuration may need to be altered in order to provide atraumatic dilation of the brain tissue if such alternate cross-sections are used. The circular cross-section and conical rounded tip of the dilating obturator illustrated in the accompanying drawings has been found to be satisfactory, and permits gentle back and forth rotation to be used during insertion to urge the blunt dissecting tip through the brain tissue.

In accordance with the above description, the dilating obturator and cannula are placed using a guide stylet which has previously been placed into the brain under image guidance. Alternative techniques are contemplated. For example, rather than using image guidance, it is contemplated that the stylet may be placed using a stereotactic headframe, such as a Leksell frame (Elekta, of Sweden) or a GTC frame (Radionics, Burlington, Mass.). In a further alternative using such a headframe, the dilating obturator and cannula may be placed using such a headframe and eliminating the need for the stylet. Such stereotactic headframes and associated methods of approaching target tissue within the brain along a predetermined trajectory are shown and described in Cosman U.S. Pat. No. 6,331,180.

In the farther alternative embodiment shown in FIG. 4, the dilating obturator equipped with image guidance means mounted directly to the obturator may be inserted under guidance without the use the stylet of the first embodiment. In using the embodiment of FIG. 4, the scalp and skull are opened in a traditional manner. Once access to the brain is established by opening and retracting the dura, dilating obturator 56 with cannula 52 mounted onto shaft 60 is urged through the brain tissue, atraumatically spreading the brain tissue until the target tissue is reached. As the dilating obturator is advanced, the position of the obturator may be checked using the image guidance system. Preferably, the obturator is pre-calibrated to the image guidance system. See, for example, Costales U.S. Pat. No. 5,921,992. In this regard, it is also contemplated that the image guidance means could be mounted to the cannula, but such an approach is less preferred because the image guidance means would remain attached to the cannula during surgery or the image guidance means would need to be removed from the cannula prior to surgery, adding another step to be performed. Attaching the image guidance means to the obturator accomplishes the objective of guiding placement of the cannula while also conveniently removing the image guidance means from the surgical field with the obturator after the cannula is placed so that the image guidance means does not obstruct the operative field.

Current image guidance systems superimpose an image of the instrument upon a pre-operative image of the patient s skull. As imaging techniques and equipment improve, it is contemplated that real time imaging will be available. Such real-time imaging techniques will be particularly useful with the techniques of the present method, as it will be possible to observe the position of the dilating obturator in relation to the real-time image of the brain structures rather than by comparison to pre-operative images. The infrared image guidance reflectors shown in the first and second embodiments are used in connection with known optical image guidance systems. Such optical image guidance systems require a direct line of sight between the image guidance balls and the camera of the image guidance system. While applicants have successfully used optical image guidance in the surgical methods described herein, it is contemplated that magnetic image guidance may be particularly well suited for use in the present method. As the name implies, a magnetic image guidance system uses magnetic forces to detect the position and orientation of the instrument. Because no direct line of sight is required, the magnetic position sensors may be detected even while positioned within the skull. It is therefore contemplated that one or more magnetic position sensors may be positioned at or near the tip of the dilating obturator so that the position of the tip may be more directly detected and displayed. One electromagnetic guidance system is available from the Visualization Technologies division of GE Medical Systems. Compare Ferre U.S. Pat. Nos. 5,676,673, 5,800,352, 5,803,089, 5,829,444, 5,873,822, 5,967,980, 6,175,756, 6,341,231, and 6,445,944.

While the preferred method utilizes image guidance to guide insertion of the dilating obturator and, hence, placement of the cannula, it is understood that it is possible to insert the dilating obturator without image guidance. Thus, the third embodiment of FIG. 5 consists only of a dilating obturator and cannula assembly without any associated image guidance apparatus. In use, the dilating obturator 76 with cannula 72 over the shaft 80 is inserted under direct visualization through brain tissue until the blunt obturator tip is adjacent to the target tissue. An experienced surgeon also may find it useful to inspect pre-operative images displayed on the monitor simultaneous with insertion of the obturator so that the surgeon may compare the pre-operative image to what is visible during insertion of the obturator under direct visualization. After the obturator and cannula of FIG. 5 has been inserted, the obturator is removed as in prior embodiments to leave the cannula in place as shown in FIG. 9.

Thereafter, surgery is preformed through the cannula. After surgery, the cannula is removed and the dura, skull and scalp are closed in a traditional manner.

The techniques described herein are particularly useful to access tumors, cysts or other conditions which might otherwise be considered inoperable or might require much more invasive transcranial surgery to remove a larger portion of the skull and retract a substantial amount of brain tissue. The techniques described herein using dilating obturator and cannula permit brain surgery to be performed in a less invasive manner through an opening in the skull that is substantially smaller then otherwise possible, on the order of a 2 cm to 4 cm in diameter rather than a much larger opening for more traditional surgical techniques.

What is claimed is:

1. A method of performing brain surgery comprising:
identifying target tissue located within the brain;
accessing the brain through an opening formed through the skull;
advancing a blunt dilating obturator into said brain tissue with a cannula mounted over said dilating obturator until the blunt dilating obturator is positioned adjacent the target tissue.
removing the blunt dilating obturator leaving the cannula in place to provide access to the target tissue.

2. The method of claim 1 wherein said step of advancing the blunt dilating obturator further comprises atraumatically dilating brain tissue.

3. The method of claim 1 wherein said step of advancing the blunt dilating obturator and cannula further comprises atraumatically spreading brain tissue until the brain tissue surrounds the cannula.

4. The method of claim 1 further comprising performing surgery on the target tissue through the cannula.

5. The method of performing brain surgery according to claim 4 further comprising inserting an endoscope into the cannula and performing surgery on the target tissue while visualizing the target tissue on a monitor.

6. The method of claim 1 further comprising breaking up and removing said target tissue through the cannula.

7. The method of claim 4 further comprising the step of removing the cannula from the brain after performing surgery.

8. The method of claim 4 further comprising the step of monitoring brain function during surgery.

9. The method of claim 1 further comprising the steps of providing a transparent cannula and viewing brain tissue through the wall of the cannula.

10. The method of claim 1 wherein said step of advancing the blunt dilating obturator until the blunt dilating obturator is positioned adjacent target tissue further comprises advancing the blunt dilating obturator under image guidance.

11. The method of claim 1 wherein said step of advancing the blunt obturator and cannula farther comprises advancing a blunt obturator and cannula having a cannula diameter of from about 10 mm to 15 mm and a cannula length of from about 2 cm to 6 cm.

12. A method of performing brain surgery comprising:
identifying a target tissue located within the brain;
accessing brain tissue through an opening formed in the skull;
directing a narrow probe through brain tissue until the probe is adjacent the target tissue;
advancing a blunt dilating obturator into the brain tis sue over the probe with a cannula disposed over the dilating obturator to gently dilate brain tissue until the obturator is located adjacent the target tissue;
removing the probe and the obturator, leaving the cannula in place to provide access to the target tissue.

13. The method of claim 12 further comprising performing surgery on the target tissue through the cannula.

14. The method of claim 12 further comprising the step of placing an endoscope within the cannula to view the brain tissue adjacent the end of the probe.

15. The method of claim 14 further comprising performing surgery on the target tissue through the cannula while observing a display of the image of the target tissue from the endoscope.

16. The method of claim 12 further comprising breaking up and removing the target tissue through the cannula.

17. The method of claim 12 further comprising the step of removing the cannula from the tissue after performing surgery through the cannula.

18. The method of claim 12 further comprising the step of monitoring brain function during surgery.

19. The method of claim 12 further comprising the step of providing a transparent cannula and viewing brain tissue through the wall of the cannula.

20. The method of claim 12 wherein the step of directing the probe through brain tissue further comprises directing the probe under image guidance.

21. The method of claim 12 wherein said step of advancing the blunt dilating obturator further comprises advancing the blunt dilating obturator under image guidance.

22. The method of claim 12 wherein said step of advancing the blunt dilating obturator further comprises atraumatically dilating brain tissue.

23. The method of claim 12 wherein said step of advancing the blunt dilating obturator and cannula further comprises atraumatically spreading brain tissue until the brain tissue surrounds the cannula.

24. The method of claim 12 wherein said step of advancing the blunt obturator and cannula further comprises advancing a blunt obturator and cannula having a cannula outer diameter of from about 10 mm to 15 mm and a cannula length of from about 2 cm to 6 cm.

25. Apparatus for accessing brain tissue comprising: an obturator and cannula assembly including
a dilating obturator having an atraumatic dilating tip and a shaft portion;
a cannula disposed around the dilating obturator shaft portion.

26. The apparatus of claim 25 wherein the atraumatic dilating tip is substantially conical.

27. The apparatus of claim 25 wherein the dilating obturator shaft portion is substantially cylindrical.

28. The apparatus of claim 25 wherein the dilating obturator has a longitudinal passage configured and dimensioned to receive an endoscope such that the endoscope is aligned with the optical window.

29. The apparatus of claim 25 wherein the dilating obturator further includes a handle portion, the handle portion and the dilating tip being disposed at opposite ends of the shaft portion.

30. The apparatus of claim 25 wherein said cannula is transparent.

31. The apparatus of claim 25 wherein the cannula has an inner diameter of from about 10 mm to about 15 mm.

32. The apparatus of claim 25 wherein the cannula has a length of from about 2 cm to about 6 cm.

33. The apparatus of claim 25 wherein the cannula has a smooth outer surface.

34. The apparatus of claim 25 wherein the cannula has a chamfered lead edge adjacent the obturator tip.

35. The apparatus of claim 25 further comprising image guidance means associated with the obturator and cannula assembly.

36. The apparatus of claim 34 wherein the image guidance means is mounted to the obturator handle.

37. The apparatus of claim 34 wherein the image guidance means is mounted to the cannula.

38. The apparatus of claim 34 wherein the image guidance means further comprise a plurality of infrared reflectors.

39. The apparatus of claim 34 wherein the image guidance means further comprise an electromagnetic indicator.

40. The apparatus of claim 25 wherein the dilating obturator has a longitudinal passage therethrough, and further comprising a surgical probe having a shaft configured and dimensioned to be received in the obturator longitudinal passage.

41. The apparatus of claim 40 wherein the longitudinal passage through the dilating obturator is configured and dimensioned to receive a probe shaft of approximately 3 mm in diameter.

42. The apparatus of claim 40 further comprising image guidance means associated with the surgical probe.

43. The apparatus of claim 42 wherein the image guidance means comprises an optical image guidance means.

44. The apparatus of claim 42 wherein the image guidance means comprises a plurality of infrared reflectors.

45. The apparatus of claim 42 wherein the image guidance means comprises an electromagnetic image guidance means.
